(19) United States
(12) Patent Application Publication
(10) Pub. No.: US 2024/0081827 A1
(43) Pub. Date: Mar. 14, 2024

(54) SURGICAL BACKGROUND DEVICE

(71) Applicants: Theodora PAPAVASILIOU, Oxford (GB); Stelios CHATZIMICHAIL, Oxford (GB); Ankur KHAJURIA, East Grinstead (GB); Joon Pio HONG, Seoul (KR)

(72) Inventors: Theodora PAPAVASILIOU, Oxford (GB); Stelios CHATZIMICHAIL, Oxford (GB); Ankur KHAJURIA, East Grinstead (GB); Joon Pio HONG, Seoul (KR)

(21) Appl. No.: 18/270,403

(22) PCT Filed: Dec. 30, 2021
(86) PCT No.: PCT/GB2021/053443

§ 371 Date: Jun. 29, 2023

(30) Foreign Application Priority Data
Dec. 30, 2020 (GB) 2020734.6

(51) Int. Cl. A61B 17/11; A61B 17/04; A61M 1/00

(52) U.S. Cl. CPC A61B 17/11; A61B 17/0482; A61M 1/71; A61B 2017/00526

(57) ABSTRACT

An anastomosis background device for surgical work-holding during the suturing of a vessel, the device comprising: a base for placement on a surgical site; at least one attachment point provided on the base for attaching at least one clamp for clamping a vessel to be sutured; and a guide structure provided on the base and positioned relative to the at least one attachment point for guiding a suture thread used in the suturing of the vessel to be sutured.

### FIELD OF INVENTION

The present invention relates to methods and apparatus and more particularly to methods and apparatus for use in surgery, and still more particularly to anastomosis surgery, and to surgical background devices, work-holding devices, and aspiration/irrigation devices for use in such surgery.

### BACKGROUND

A common difficulty encountered by surgeons when performing surgery is poor visibility at the surgical site and the high level of dexterity required for surgical procedures. This is a particular problem in microsurgery and super-microsurgeries where both of these factors are accented by the small scales involved. Surgical requirements may further impair visibility of the surgical site and contribute to the degree of dexterity required of the surgeon. For example, a number of surgical procedures require irrigation of the surgical site such irrigation can reduce visibility and may also require an assistant to operate a suctioning hose at or near the surgical site. This may further decrease the space available for the surgeon to work and hamper visibility.

Anastomosis procedures represent a particular challenge.

During anastomosis procedures, in addition to the reduction in visibility caused by irrigation, contrast between the vessel and the surgical site may be poor. Irrigation may further hamper visibility and make it still more difficult to distinguish details of the vessel being sutured. There is therefore a need to provide an apparatus and method that improves visibility and contrast during surgery and which assists the surgeon during surgical procedures.

Aspects and examples of the invention are set out in the claims and aim to address at least a part of the above described technical problem, and other problems by providing an apparatus to improve visibility and contrast at the surgical site and to reduce the level of dexterity required for surgical procedures.

### SUMMARY

Aspects and examples of the invention are set out in the claims. Further examples of the disclosure are described herein for understanding the claims and for providing optional further refinements and modifications of their teaching.

In an aspect there is provided an anastomosis background device for surgical work-holding during the suturing of a vessel. One such device comprises: a base for placement on a surgical site, the base carrying at least one attachment point for attaching, to the base, at least one clamp for clamping a vessel to be sutured. A guide structure is carried by the base and positioned relative to the at least one attachment point for guiding a suture thread used in the suturing of the vessel to be sutured when, in use, it is held by a clamp or clamps fixed to the attachment point.

An aspect of the disclosure provides an anastomosis background device for aiding the suturing of a vessel. One such device comprises: a base for placement on a surgical site to provide a background for said suturing, the base comprising: a drain for draining fluid from the background; and a suction port in fluid connection with the drain for aspirating fluid from the drain.

The device carries at least one attachment point on the base for attaching at least one clamp for clamping a vessel to be sutured; and a guide structure provided on the base and positioned relative to the at least one attachment point for guiding a suture thread used in the suturing of the vessel to be sutured.

The guide structure may comprise a runner configured to constrain the position of the suture whilst enabling the suture to slide along the runner thus to permit traction of the suture whilst constraining its position (e.g. its height above the base at a fixed location relative to the base).

The runner may be implemented in a variety of ways—for example it may provide to constrain height the runner typically comprises an upwardly facing surface, such as might be provided by the side of a recess such as a groove, or by a face of a protrusion such as a ridge. For example each runner may comprise a groove in a corresponding one of the poles. Such grooves may extend circumferentially around said corresponding pole.

The guide structure may comprise a plurality of runners each disposed at a different position, for example the different positions may comprise a plurality of heights above the base and/or a plurality of lateral locations on the base.

One way to provide such arrangements is for the guide structure to comprise a plurality of poles one disposed at each of said plurality of lateral locations.

The lateral locations of the poles may be selected relative to the location of the attachment point to enable traction to be applied, to a vessel held in the clamps to be sutured, in one of a set of defined directions. For example the poles may be positioned so that traction can be applied in at least two different directions (e.g. mutually non-aligned directions).

The runners may be provided at three lateral locations, which may correspond to the vertices of a triangle on the base. For example there may be three poles, one disposed at each of these three locations. A side of said triangle may be aligned with a clamped position of the vessel to be sutured—for example aligned with a direction of separation of two clamps attached to the attachment point.

The guide structure may be removable from the base, for example it may comprise a frangible portion, e.g. a joint between the guide structure and the base that is weaker than the base to permit removal of all or part of the guide structure without damage to the base.

The drain may comprise an aperture in the base and a drainage channel linking the aperture to the suction port.

The suction port may comprise a hose barb, e.g. for connecting a suction hose to the port.

To provide a clear background for surgical work, the colour of the base may be selected to contrast with the colour of the surgical site and/or the vessel to be sutured. Examples of preferred colours include blue and green.

The base may comprises a plurality of stay holes through which a stay suture may be secured. This may help to hold the device in place during a surgical procedure and permit its simple removal once the procedure is complete.

The plurality of stay holes may be positioned at the periphery of the base, for example at its edges and/or its corners.

The device may be plastic and/or may be flexible. In some cases a rigid material, such as a rigid plastic, may be used.

The device may be formed by processes of additive manufacture, such as 3D printing. Embodiments of the disclosure may provide a machine readable map or instructions configured to enable a physical representation of the device to be produced by 3D printing.

The devices described and claimed herein may further comprise at least one clamp for holding a vessel to be sutured and configured for attachment to the at least one attachment point of the base. The device may comprise a first said clamp attached at a first attachment point and a second said clamp attached at a second attachment point. These attachment points may comprise separate locations on a single attachment “point” which may be provided by a groove and/or a ridge. It will thus be appreciated in the context of the present disclosure that the term attachment point is not to be understood in narrow mathematical or geometric terms.

The first and second clamps may be arranged such that a first part of vessel may be clamped in the first clamp and a second part of a vessel may be clamped in the second clamp such that the first and second parts are clamped in proximity for suturing of an end of the first part of the vessel and an end of the second part of the vessel together.

An embodiment provides a method of suturing a lumen of a human or animal body, the method comprising: placing a base of a tool having a guide structure and a clamp at a surgical site; clamping the lumen in the clamp; providing a first stitch of suture thread in the anterior wall of the lumen (e.g. the side of the lumen presented to the surgeon); securing the suture thread to the guide structure; tensioning the suture thread against the guide structure such that the next stitching location is presented to the user; providing a next stitch at the next stitching location; and repeating the securing of the suture thread, tensioning and providing steps above to complete a suture of a lumen.

The method may comprise attaching an aspiration hose to a suction port provided in the base of the tool to aspirate fluid from a drainage channel in the base of the tool.

The method may comprise securing the tool to the surgical site by providing stay sutures through securing holes in the base and into tissue at the surgical site.

It will be appreciated in the context of the present disclosure that these and other methods may be implemented by suitable configurations of the apparatus described and claimed herein.

### BRIEF DESCRIPTION OF DRAWINGS

Some practical implementations will now be described, by way of example only, with reference to the accompanying drawings in which:

FIG. 1 shows a schematic view of an anastomosis background device;

FIG. 2 shows a cross-sectional view of the anastomosis background device of FIG. 1;

FIG. 3 shows a schematic view of another anastomosis background device; and

FIG. 4 shows a schematic view of yet another anastomosis background device.

In the drawings like reference numerals are used to indicate like elements

### SPECIFIC DESCRIPTION

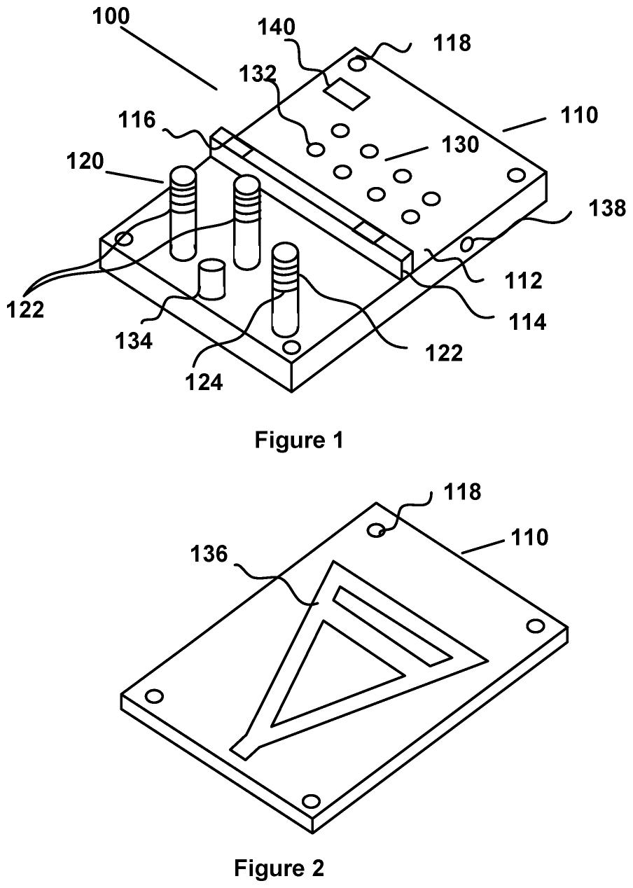

FIG. 1 shows an anastomosis background device 100 for surgical work-holding during the suturing of a vessel (e.g. for holding the vessel to be sutured).

The device comprises a flat rectangular base 110, a guide structure 120, a drainage system 130, and clamp attachment structure 114.

Typically, during anastomosis surgery two ends of a vessel are each clamped by a surgical clamp and then sutured together by a surgeon.

The device of the present application seeks to aid this process by providing an effective background to the surgical work and to provide a structural aid for the act of suturing.

Specifically, during an anastomosis, the device 100 is arranged at a surgical site by the operator so that the base 110 provides a background to the anastomosis and so that the guide structure 120 may be used to aid in the act of suturing.

To provide an effective background the colour of the base 110, and in particular a top surface 112 of the base, is selected to contrast with the colour of the surgical site and is provided with a drainage system 130.

The colour of the base 110 is selected depending on the type of surgery being performed and the characteristics, such as colour, of the surgical site. For example, in many microsurgical operations such as arterial and venous anastomoses, the microvascular background may be either blue or green.

During surgical procedures the surgical backgrounds can become obscured by liquid provided to the surgical site e.g by irrigation used for irrigation of the surgical site or surgical material for example blood, tissue, bodily fluid. Liquids used for irrigation may include isotonic saline or sodium heparin (anti-coagulant) solution. To prevent obscuration, a drainage system 130 is provided in the base 112 that facilitates and automates the removal of fluids from the base 110 and its proximity. The removal of surgical material prevents the base 110, and therefore the background, from becoming obscured by fluids during surgery.

An example of a drainage system 130 is shown in FIG. 1 and FIG. 2 and will be described now in detail with reference to these figures.

As can be seen in FIG. 1, an array of apertures, drain holes 132, are provided in the top surface 112 of the base 110. The drain holes 132 extend through the top surface 112 of the base and connect with a drainage channel 136, or number of drainage channels, provided in the interior of the base 110.

The drainage channels 136 extend through the interior of the base 110 and connect through the top surface 112 to a suction port 134, in this case a hose barb, provided in the top surface 112 of the base.

In the present example, the hose barb 134 is positioned on the top surface 112 of the base 110 at a peripheral end opposite to the drain holes 132 in the top surface 112.

The hose barb 134 positioned at the peripheral edge of the top surface 112 is easily accessible during surgery and does not to obstruct the use of the guide structure 120.

The suction port 134 could also be placed at other suitable locations on the base for example on the side of the base 110.

The hose barb 134 provides a suction port to which a suctioning device can be attached, for example a surgical suction hose, thereby automating the removal of fluids or surgical material from the base 110 of the device.

The automated removal of the fluids or surgical material advantageously allows the surgeon to focus on the surgery. Also by not requiring manual removal of fluids, for example by an assistant using a surgical hose, device 100 reduces the level of dexterity required to perform the anastomosis.

Surgical material collecting on the top surface 112 of the base 110 will drain through the drain holes 132 into the drainage channels 136 and can be aspirated from the drainage channels via the hose barb 134 using a suctioning device.

In use, with a suctioning device attached to the hose barb 134 and actively aspirating, a suctioning force is applied in the proximity of the drain holes 132, actively suctioning fluid and surgical material in the proximity of the drain holes into and through the drainage channels 136 where it is then aspirated from the device 110 via the hose barb 134.

Additional apertures, drain holes 138, connected to the internal drainage channels are provided through the lateral aspects of the base. The additional drain holes allow fluid collecting around the edge of the base to be suctioned into the drainage channels.

A clamp attachment structure 114 with attachment points 116 for surgical clamps is provided on the top surface 112 of the base 110 of the device of FIG. 1. Whilst it is possible to implement a device without any such attachment points, securing surgical clamps to the base offers improved stability of the clamped vessel during surgery, improved alignment of the clamped ends of the vessel, and allows the surgeon to focus on suture technique. The example clamp attachment structure 114 shown in FIG. 1 will now be described in detail.

As can be seen in FIG. 1, a raised structure 114 extends across a width of the top surface 112 of the base 110 bisecting the top surface 112. The raised structure is a cuboid, having an exposed flat upper surface that is parallel with the top surface 112 of the base. The raised structure is formed as one piece with the base of the device. Provided along the upper surface of the raised structure 114 are two cut-away portions 116 into which an end of a surgical clamp can be inserted, and which acts to secure the clamp end. The cut-away portions 116 form an angled slit in the upper surface extending down into the raised structure. The angle of the slit is 45° relative to the base of the device and is directed away from the drain holes 132. The dimensions of the slit are selected to accommodate the ends of surgical clamps, for example the slit may be about 1.5 mm wide and 4 mm deep.

The cut-away portions are aligned with respect to each other so that the inserted clamps are also substantially aligned and parallel. The flat upper surface of the raised structure 114 provides a working surface on which the vessel being sutured may be rested during the procedure.

During an anastomosis procedure an end of a clamp clamping one end of a vessel to be sutured is inserted into and secured in one of the cut-away portions 116 and a clamp clamping another end of the vessel to be sutured is inserted and secured in the other of the cut-away portions 116. Once each of the clamps has been secured in the respective cut-away portions, the clamped ends of the vessel are in proximity to one another enabling the ends to be sutured together.

The device also comprises a guide structure 120 to aid in the act of suturing during an anastomosis surgery.

An example of a guide structure 120 will now be described in more detail and with reference to FIG. 1.

As can be seen in FIG. 1, a guide structure 120 is provided by three cylindrical poles 122 extending from the top surface 112 of the base 110 of device 100. The poles may be circular, and may have a diameter of between 2 mm and 6 mm, for example about 4 mm.

The outer surface of each of the cylindrical poles may comprise a series of circumferential grooves, such as a helical thread. These and other arrangements of grooves can provide a series of recessed runners 124, each groove providing a “runner” channel into which a suture thread may be slotted. The size of these grooves is selected to allow a suture thread to be constrained within the groove whilst allowing the thread to pass through the runner. For example, a width of the groove in the thread of 0.04 mm may be used to accommodate a suture thread having a diameter of similar dimensions 0.035 or smaller. The grooves typically are comparable in size to the size of the sutures—this may maximise contact points. An exemplar thread pitch that can be used is 0.25 mm. The suture may be provided in a kit with the device and the size of the provided suture may match the size of the grooves.

During the suturing of a vessel, securing the suture thread in a groove/runner of poles 122 allows a tension to be applied to the thread and as a result of this tension, traction is applied to the vessel that is being sutured.

The poles 122 are positioned at different lateral locations on the top surface 112 of the base 110 relative to clamp attachment structure 114. The lateral positioning of poles 112 and the vertical positions of grooves on them relative to a vessel to be sutured held in the attached clamps allows a number of different tensioning directions to be applied to a suture thread when the thread is secured in and runs through a groove. Lateral tension is maintained on the suture thread by tensioning it against the pole. Vertical tension is applied on the suture thread by a wall of the groove of the runner, in this case a lower wall of the groove as the vessel to be sutured is positioned below the runners. Applying tension to the suture thread in different directions applies traction to the vessel allowing the vessel to be manipulated in a number of different ways thereby aiding in the act of suturing. In particular, tension can be applied using the guide structure 120 to rotate the vessel being sutured to improve the surgeons view of the suture. For example, during an anastomosis applying tension to a suture thread restrained in a runner will result in the vessel to be sutured being pulled, laterally and vertically, in the direction of the runner.

The three poles 122 of the present example are laterally positioned in a triangular formation on the top surface of the base. The poles 122 are positioned relative to the mid-point of the raised structure 114. The poles 122 are arranged on one side of the bisected top surface 112 at angular intervals of 45°. The poles 122 are at positioned at angles of approximately 45°, 90° and 135° relative to the mid-point of the raised structure 114. The pole at 90° is offset from the poles at the 45° and 135° degree angle in a lateral direction toward the raised structure thereby forming a substantially triangular formation with the other two poles.

The threaded portions 124 of each of the three poles 122 extends above the upper surface of the raised structure 114 and provides a structure that can be used to tension a suture thread at a number of different vertical angles. Applying tension to the suture thread in different vertical directions applies traction to the vessel being sutured in different vertical directions allowing the vessel to be manipulated in another number of different ways and thereby aiding in the act of suturing.

The vertical tensioning in combination with the lateral tensioning allows for complex manipulations of the vessel being sutured. Furthermore, the poles 122 may be used in combination to provide further tensioning possibilities to a suture thread to achieve a desired manipulation of the vessel. For example, the suture thread may be placed in the runners 124 of two or more of the poles 122 and tensioned using the resistance from both of the poles 122.

The poles 122 of the guide structure 120 are breakable from the base. This allows the user to remove one or more of the poles 122 to increase the space available at the surgical site.

A number of other features of the device provide further functionality and will now be described with reference to FIGS. 1 and 2.

Holes 118 provided at the corners of the rectangular base 110 are used to secure the device 110 to the surgical site using stay sutures through the holes.

Furthermore, during an anastomosis procedure it is often necessary for a suture needle to be cleaned or for the suture needle to be retained in place, for example between suture stiches. Provided on the top surface of the base is an abrasive patch 140 for retaining and/or cleaning a suture needle. The patch is made from a fibrous material and in this example from the hook part of a hook and loop fastener.

The top surface of the base 110 has surface grid which can be used as a measurement tool and which reduces light reflection. Light reflection is known to contribute to eye fatigue.

The surface grid in this example is formed from the top surface 112 of the base 110. For example, a series of parallel grooves with a width of 1 mm extending across the top surface and spaced at 1 mm intervals.

The device 100 may have a monolithic construction. For example, at least the base of the device, the drain and its suction port may be provided by a single piece of material. Generally the base has a top surface on which, in use, a vessel to be sutured is positioned for suturing. An aperture for collecting and aspirating fluid is provided in the top surface of the base, and the suction port comprises a hose barb. The aperture, the drain through the base and the suction port, optionally including this hose barb may all be integrated in the sense that they may all be provided by the same single unitary piece of material, such as plastic. This structure may be made by additive manufacture, or by moulding. It can thus be seen that the base 110 may consist of a single unitary piece through which the drainage channels 136 of the drainage system 130 extend. The single unitary piece does not have any joins which may increase the effectiveness of suction provided at the drain holes 132.

The device is 3D printed as one piece and is manufactured using a plastic for example Polylactic Acid variant (tough-PLA: pla− pro+ from RS). Other plastics may also be used such as ABS, PEEK, polypropylene. The device is rigid which, for certain surgical situations, advantageously allows base of the device to be used to hold back tissue at the surgical site. The thermoplastic device can be autoclaved and is suitably radiopaque that X-ray films can be used to locate the device.

Some surgical situations may require or benefit from a flexible device for example where space at the surgical site may be limited or in situations where the shape of the device does not allow placement at the surgical site. The device may therefore alternatively be made from a flexible polymer such as thermoplastic elastomers TPE, and/or polyurethane.

The device of FIGS. 1 and 2 will now be described in use in an anastomosis microsurgery on a blood vessel.

During an anastomosis microsurgery, the base 110 is placed at the surgical site with the top surface 112 and the poles 122 of the guide structure 120 toward the user. Stay sutures are provided in the holes 118 at the corners of the base to secure the base 110 to the surgical site. A surgical hose is attached to the hose barb 134 on the top surface 112.

The ends of the blood vessel to be sutured are clamped, using surgical clamps, and the ends of the clamps are attached to the base of the device at the attachment points provided by the cut-out portions 116.

After attaching the clamps, the ends of the vessels are arranged on the raised structure 114 in proximity to one another. By virtue of the slit angle of the cut out portions 116, the secured clamps are angled toward the three poles 122 of the guide structure 120.

The base 110 is therefore positioned between the ends of the vessel to be sutured and the surgical site thereby providing a background to the procedure. The colour of the base acting as an effective contrast compared to the colour of the surgical site and vessel.

The surgical hose is switched on to provide suctioning to the drainage system 130. Surgical material and irrigation fluid in proximity to the drain holes of the device 100 is suctioned into the drainage channels 136 and removed via the hose barb 134 by the suction hose thereby maintaining an effective background for the procedure by preventing obscuration of the top surface 112 of the base 110.

The operator provides a first stitch in an anterior wall of the lumen of the blood vessel. After the first stich has been made the operator may wish to rotate the vessel to achieve better visibility of the location for the next stitch.

Normally, this would require the operator to manipulate the blood vessel manually, however by using the guide structure 120 the operator can apply tension to the thread and cause rotation of the vessel. Specifically, following the first stitch, the operator selects a runner in one of the three poles in which to secure the suture thread depending on the direction of tension required. The selection of the pole dictating the lateral component of tension applied to the suture thread and the selection of runner, in this case a particular groove of the selected threaded pole, dictating the vertical component of the tension applied to the suture thread. Once the suture thread has been secured in the selected runner, tension is applied to the suture thread causing traction to be applied to the vessel. The traction on the vessel from the suture thread causes the vessel to rotate and present the next stitching location to the operator. The operator then applies the next stitch at the next stitching location and repeats the process until the ends of the vessels have been sutured, and when appropriate after each stitch securing the suture thread in a selected runner that provides the required traction to rotate the vessel to the appropriate position to visualise the location for the next stitch.

The operator may simultaneously secure the suture thread in one or more runners in one or more poles, for example the suture thread could secure a stitch part to a first runner on a first pole, and part way through leave that stitch part then use a second runner on a second part to add a further part of the same stitch.

Between stitches or stitch parts, the needle may be cleaned and/or retained on the abrasive patch 140 on the top surface 112 of the base. Retaining the needle in this way advantageously prevents the needle from blocking the drain holes 132.

Once the suture is completed, the clamps are removed from the attachment points 116, the stay sutures and suction hose are removed and the device is removed from the surgical site and discarded.

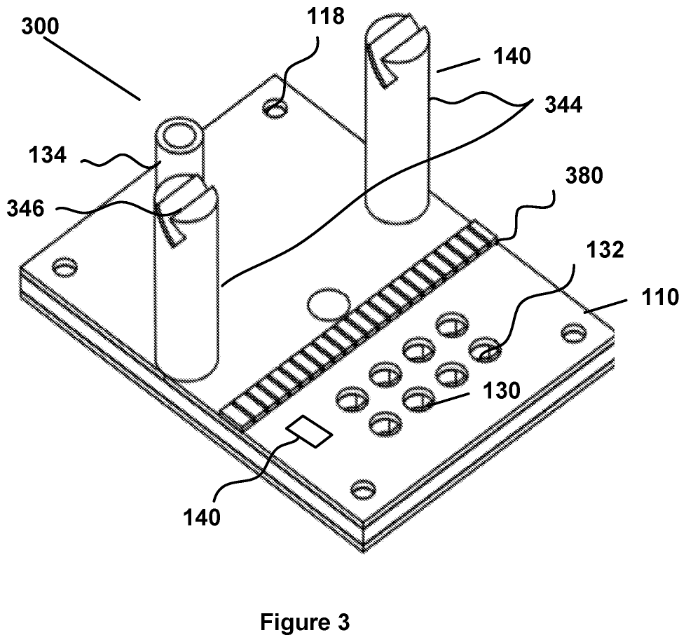

FIG. 3 shows another example of an anastomosis background device 100 for surgical work-holding during the suturing of a vessel similar to that of FIGS. 1 and 2.

The device 300 comprises a rectangular base 110, a guide structure 120, a drainage system 130, and clamp attachment structure 114.

The base of the device is substantially the same as that of FIG. 1 with the exception of the clamp attachment structure 344 and the inclusion of a frangible section 380.

As with the device 100 of FIGS. 1 and 2 the drainage system 130 comprises drain holes, drainage channels and a suction port. The upper surface of the base has an array of drain holes 132 that connect to drainage channels 136 in the interior of the base. A suction port 134 is provided in the upper surface 112 at a peripheral end of the rectangular base 110. The drainage channels 136 extend through the interior of the base 110 and connect through the top surface 112 to the suction port 134.

The clamp attachment structure 114 of device 300 comprises two columns 344 that protrude from the upper surface 112 of the base 110. At the exposed end of each of the columns 346 is a clamp attachment point in the form of a cut-away portion 346.

An end of a surgical clamp or a connecting bar of a pair of connected surgical clamps can be inserted and retained in the cut-away portion 346 of the column 346. The cut-away portion 346 is an angled slit in the top of the column extending down into the column. The angle of the slit is 45° relative to the base of the device and is angled away from the array of drain holes 132.

A frangible section 380 is provided in the base 110 across the width of the midpoint of the base 110 between the array of drain holes 132 and the clamp attachment structure 114. The frangible section 380 provides a structural weak point in the device 300 to enable the base 110 to be broken into two sections along the frangible section 380.

The frangible section 380 thereby allows a user to alter the dimensions of the device in circumstances where a smaller device is needed, for example for use in a surgical site in which the entire device does not fit. The user breaks the base 110 of the device along the frangible section 380 retaining the section of the device having the drain holes 132 and discarding the other section.

The retained section has additional drain holes along the exposed edge due to the now exposed interior drainage channels in the base of the device. Breaking along the frangible section exposes an interior suction inlet on the exposed edge of the retained section.

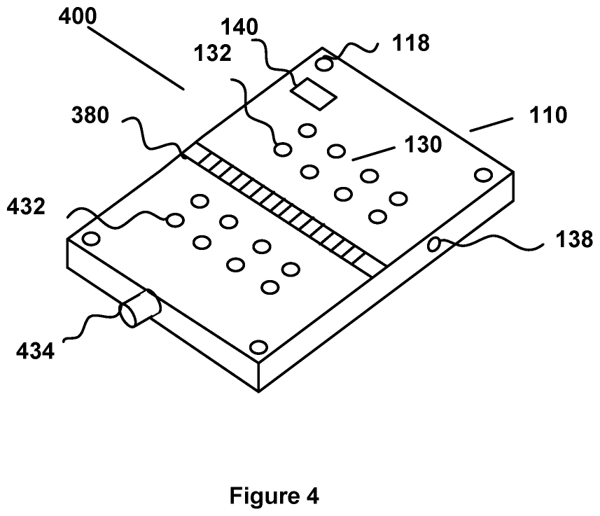

FIG. 4 shows another example of a surgical background device, similar to that of FIGS. 1, 2 and 3 but without a guide structure 120 and clamp attachment structure 114.

The device 400 of FIG. 4 has two arrays of apertures, drain holes 132 and 432, in the top surface 112 of the base 110. As with the device 100 of FIGS. 1 and 2 the drain holes 132 of device 400 connect to drainage channels 136 in the interior of the base as do drain holes 432.

A suction port 434 is provided on a lateral aspect of the device rather than the top surface of the base. The suction port 434 is connected to the drainage channels through the side of the base 110. A suctioning device can be attached to the suction port 434 to aspirate the drainage channels and to provide a suctioning force in the proximity of the drain holes. Further apertures, drain holes 138, are provided in the lateral aspects of the base and are connected to the drainage channels.

A frangible section 380 is provided across the width of the midpoint of the base between the two arrays of drainage holes 132, 432. Similar to the device 300 of FIG. 3, the frangible section 380 provides a structural weak point in the device 400 to enable the base 110 to be broken into two sections along the frangible section 380.

The frangible section 380 thereby allows a user to alter the dimensions of the device in circumstances where a smaller device is needed. The user breaks the base of the device along the frangible section 380 retaining the section of the device having the suction port 434 and discarding the other section.

The retained section has additional drainage holes along the exposed edge due the now exposed interior drainage channels in the base of the device and may be used in similar manner to devices 100 and 200 to provide a background and suction at the surgical site.

In this example the device does not have a clamp attachment structure 114. Therefore, during an anastomosis instead of securing the clamps clamping the ends of the vessel to the base 110 the clamps are simply arranged unsecured in a similar position to the position that they would be if clamp attachment structure were present. That is to say the clamps are arranged so that the base 110 is positioned between the vessel to be sutured and the surgical site, thereby providing a background to the procedure.

The devices 100, 300, and 400 are envisaged to be single use surgical devices that are discarded after they have been used.

Whilst no specific dimensions have been provided for the devices described above they may be dimensioned for use in a wide range of surgical procedures. In particular, the devices may be dimensioned for use in microsurgical and super-microsurgical procedures and dimensioned to be used with surgical equipment used in microsurgical and super microsurgical procedures such as micro needles, micro suture thread and surgical clamps.

Furthermore, the devices may be made from radiopaque material but may also have radiopaque markers added to them to allow them to be located using radio imaging techniques.

The term clamp attachment structure has been used throughout the description. The term refers to a structure having one or more attachment points to which surgical clamps can be attached to and retained by the structure. Clamp attachment structures that have static attachment means or clamp attachment structures that have mechanical attachment means to retain clamps are envisaged.

Attachment points may be continuous for example a channel or slot or discrete separate attachment points as described above.

The guide structure 120 of device 100 and 300 may be broken off of the base 110. Alternatively, the guide structure may be made to be reversibly detachable from the base for example using a screw thread allowing the poles 122 or columns 346 to be attached or detached from the device 100 as required.

The term runner has been used throughout the description and refers to a structure that is capable of restraining the vertical and lateral position of a suture thread whilst simultaneously allowing the suture thread to move through the runner. In structural terms protrusions from or recesses in the surface of a structure protruding from or recessed in the base are suitable for restraining the vertical position of a suture thread. These protrusions or recesses may take many forms.

Similarly, protrusions from or recesses in the base are suitable for restraining the lateral position of a suture thread. These protrusions and recessed may also take many forms.

Microsurgery is taken broadly to mean any surgery requiring the optical enhancement of a surgical site for example through the use of an operating microscope. Super microsurgery refers broadly to surgery on vessels with a diameter of less than 1 mm and/or which utilises super microsurgical needles which have a diameter of less than 100 microns.

Anastomosis refers to the surgical technique used to make a surgical connection between two body structures. In particular, a surgical connection between two fluid carrying vessels. The term surgical connection refers to an artificial connection which is used to connect or secure together the body structures. Commonly anastomosis is used to create or restore a fluid connection between one or more fluid carrying vessels.

During an anastomosis the vessels to be surgically connected are clamped at their ends using surgical clamps or microsurgical clamps. Microsurgical clamps are typically of the order of 20 mm in length and have jaw dimensions on the order of 4 mm×1 mm. Microsurgical clamps are made from sterilisable material for example stainless steel.

The background device may be manufactured by any appropriate method, such as injection-moulding, casting, or by additive manufacturing techniques such as 3D printing. In some such additive manufacture methods a three-dimensional model of the background device is supplied, in machine readable form, to a ‘3D printer’ adapted to manufacture the background device. As alternatives to the methods already mentioned above, this may be by additive means such as extrusion deposition, Electron Beam Freeform Fabrication (EBF), granular materials binding, lamination, photopolymerization, or stereolithography or a combination thereof. The machine readable model may comprise a spatial map of the object to be printed, typically in the form of a Cartesian coordinate system defining the object's surfaces. This spatial map may comprise a computer file which may be provided in any one of a number of file conventions. One example of a file convention is a STL (StereoLithography) file which may be in the form of ASCII (American Standard Code for Information Interchange) or binary and specifies areas by way of triangulated surfaces with defined normals and vertices. An alternative file format is AMF (Additive Manufacturing File) which provides the facility to specify the material and texture of each surface as well as allowing for curved triangulated surfaces. The mapping of the background device may then be converted into instructions to be executed by 3D printer according to the printing method being used. This may comprise splitting the model into slices (for example, each slice corresponding to an x-y plane, with successive layers building the z dimension) and encoding each slice into a series of instructions. The instructions sent to the 3D printer may comprise Numerical Control (NC) or Computer NC (CNC) instructions, preferably in the form of G-code (also called RS-274), which comprises a series of instructions regarding how the 3D printer should act. The instructions vary depending on the type of 3D printer being used, but in the example of a moving printhead the instructions include: how the printhead should move, when/where to deposit material, the type of material to be deposited, and the flow rate of the deposited material.

Embodiments of the disclosure comprise machine readable instructions configured to cause an additive manufacturing system to produce a background according to any of the examples of such described or claimed herein.

It is to be appreciated in the context of the present disclosure that, to the extent that certain methods may be applied to the living human or animal body, it will be appreciated that such methods may be applied in circumstances which do not provide any surgical or therapeutic effect. For example, such methods may be applied ex vivo, to tissue samples that are not part of the living human or animal body. For example, the methods described herein may be practiced on meat, tissue samples, cadavers, and other non-living objects.

It will be appreciated from the discussion above that the examples shown in the figures are merely exemplary, and include features which may be generalised, removed or replaced as described herein and as set out in the claims. As will be appreciated by the skilled reader in the context of the present disclosure, each of the examples described herein may be implemented in a variety of different ways.

Any feature of any aspects of the disclosure may be combined with any of the other aspects of the disclosure. For example method aspects may be combined with apparatus aspects, and features described with reference to the operation of particular elements of apparatus may be provided in methods which do not use those particular types of apparatus.

In addition, each of the features of each of the examples is intended to be separable from the features which it is described in combination with, unless it is expressly stated that some other feature is essential to its operation. Each of these separable features may of course be combined with any of the other features of the examples in which it is described, or with any of the other features or combination of features of any of the other examples described herein. Furthermore, equivalents and modifications not described above may also be employed without departing from the invention.

Other examples and variations of the disclosure will be apparent to the skilled addressee in the context of the present disclosure.

1. An anastomosis background device for aiding the suturing of a vessel, the device comprising:
a base for placement on a surgical site to provide a background for said suturing, the base comprising:
a drain for draining fluid from the background; and
a suction port in fluid connection with the drain for aspirating fluid from the drain, wherein the base consists of a single piece of material.

2. The device according to claim 1 wherein the drain comprises:
an aperture in the base; and
a drainage channel linking the aperture to the suction port.

3. The device of claim 1 wherein the drainage channel is provided through the base to the suction port.

4-37. (canceled)

38. The device of claim 1 wherein the device is of a monolithic construction.

39. The device of claim 2 wherein the base has a top surface on which, in use, a vessel to be sutured is positioned for suturing, wherein the aperture is provided in the top surface of the base.

40. The device according to claim 1 wherein the suction port comprises a hose barb.

41. The device of claim 1 comprising:
at least one attachment point provided on the base for attaching at least one clamp for clamping a vessel to be sutured; and
a guide structure provided on the base and positioned relative to the at least one attachment point for guiding a suture thread used in the suturing of the vessel to be sutured.

42. The device of claim 41 wherein the guide structure is removable from the base.

43. An anastomosis background device for surgical work-holding during the suturing of a vessel, the device comprising:
a base for placement on a surgical site:
at least one attachment point provided on the base for attaching at least one clamp for clamping a vessel to be sutured; and
a guide structure provided on the base and positioned relative to the at least one attachment point for guiding a suture thread used in the suturing of the vessel to be sutured.

44. The device according to claim 43 wherein the guide structure comprises a runner configured to constrain the position of the suture whilst permitting traction of the suture through the runner.

45. The device according to claim 44 wherein the runner comprises a recess for accommodating a suture.

46. The device according to claim 45, wherein the guide structure comprises a plurality of runners each disposed at a different position.

47. The device according to claim 46 in which the different positions comprise a plurality of heights above the base.

48. The device according to claim 46 in which the different positions comprise a plurality of lateral locations on the base.

49. The device according to claim 48 in which the guide structure comprises a plurality of poles one disposed at each of said plurality of lateral locations.

50. The device according to claim 49 in which each runner comprises a groove in a corresponding pole.

51. The device according to claim 50 wherein the groove extends circumferentially around said corresponding pole.

52. The device according to claim 48 wherein the lateral locations are selected relative to the location of the attachment points to enable the suture to apply traction to the vessel to be sutured in one of a set of defined directions.

53. The device of claim 43 comprising a drain for draining fluid from the background; and a suction port in fluid connection with the drain for aspirating fluid from the drain.

54. A tangible non-transitory computer readable storage media storing data defining a map or instructions configured, when followed by a device having 3D printing capability, to enable a physical representation of an anastomosis background device for aiding the suturing of a vessel to be produced by 3D printing, wherein the anastomosis background device comprises:
a base for placement on a surgical site to provide a background for said suturing, the base comprising:
a drain for draining fluid from the background; and
a suction port in fluid connection with the drain for aspirating fluid from the drain, wherein the base consists of a single piece of material.
